(19) United States
(12) Patent Application Publication
(10) Pub. No.: US 2010/0120917 A1
(43) Pub. Date: May 13, 2010

(54) BUTYLAMMONIUM MODULATORS OF FATTY ACID TRANSPORT

(75) Inventors: Thomas G. Gant, Carlsbad, CA (US); Manouchehr Shahbaz, San Diego, CA (US); Craig Hodulik, San Diego, CA (US)

Correspondence Address: GLOBAL PATENT GROUP - APX, 10411 Clayton Road, Suite 304, ST. LOUIS, MO, 63131, US

(73) Assignee: Auspex Pharmaceuticals, Inc., Vista, CA (US)

(21) Appl. No.: 12/615,320

(22) Filed: Nov. 10, 2009

Related U.S. Application Data
(60) Provisional application No. 61/113,239, filed on Nov. 11, 2008.

(51) Int. Cl. A61K 31/195; C07C 229/00

(52) U.S. Cl. 514/561; 562/567; 562/568

(57) ABSTRACT

The present invention relates to new butylammonium modulators of fatty acid transport, and/or removal of toxic acyl-CoA compounds, pharmaceutical compositions thereof, and methods of use thereof

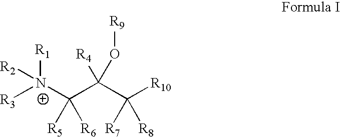

This application claims the benefit of priority of U.S. provisional application No. 61/113,239, filed Nov. 11, 2008, the disclosure of which is hereby incorporated by reference as if written herein in its entirety.

Disclosed herein are butylammonium compounds, pharmaceutical compositions made thereof, and methods modulate the transportation of fatty acids and/or removing toxic acyl-CoA compounds in a subject are also provided for, for the treatment of disorders such as hyperammonemic encephalopathy, phenylketonuric (PKU), diabetes, Peyronie's disease, Parkinson's disease, intermittent claudication, alcohol-related liver disease, dementia, Down's syndrome, male infertility, septic shock, cardiovascular shock, traumatic shock, pain associated with sciatica, hyperthyroidism, Alzheimer's disease, hypoxia-induced oxidative stress, muscle fatigue, geriatric depression, osteopenia, osteoporosis, disorders resulting from or associated with hemodialysis, oocyte cytoskeleton damage, cardiac disease, embryo apoptosis, isotretinoin induced hepatoxicity, doxorubicin induced toxicity, cancer chemotherapy induced heart damage, valproate induced hepatotoxicity, end stage renal disease, peripheral arterial disease, hepatic encephalopathy, physical fatigue, chronic fatigue syndrome, mental fatigue, primary carnitine deficiency syndromes, systematic carnitine deficiencies, secondary carnitine deficiency states, organic acidurias, leptin resistance, suboptimal energy partitioning, and disorders ameliorated by providing neuroprotection.

L-Carnitine, (3-Hydroxy-4-trimethylammonio-butanoate, Carnicor, Proxeed™, Carnitiene, Carnitor®, Carnum, Carrier, Levocarnitine, Miocor, Miotonal, and Vitacarn®) plays an important role in fat metabolism, by shuttling fatty acyl-Coenzyme A molecules from the cytosol into the mitochondria. In addition, carnitine is a strong anti-oxidant and can provide neuroprotection. Acetyl-L-carnitine (ALCAR) is pharmodynamically similar to L-carnitine, but it may have higher bioavailability. L-Carnitine and ALCAR have shown promise in treating hyperammonemic encephalopathy (Limketkai et al., J Gen Intern Me. 2008, 23(2), 210-3), diabetes (Cha Y. S., Asia Pac J Clin Nutr 2008, 17 Suppl 1, 306-8; and Rajasekar et al., Exp Diabetes Res 2007, 2007, 727-41), PKU (Sitta et al., Cell Mol Neurobiol epub Sep. 24, 2008), Peyronie's disease (Smith et al., Int J Impot Res 2008, 20(5), 445-59), Parkinson's disease (Zhang et al., J Cell Mot Med epub Jun. 20, 2008), intermittent claudication (Meru et al., Atherosclerosis 2006, 187(2), 221-37), alcohol-related liver disease (Sachan et al., Am J Clin Nutr 1984, 39, 738-744; and Sachan et al., Nutr Rep Int 1983, 27, 1221-1226), dementia (Ames et al., Ann NY Acad Sci 2004, 1033, 108-16; and Hudson et al., Cochrane Database Syst Rev 2003, 2, CD003158), Down's syndrome (De Falco et al., Clin Ter 1994, 144, 123-27), male infertility (Zhou et al., Asia Pac J Clin Nutr 2007, 16 Suppl 1, 383-90), septic shock (Winter et al., Br J Cancer 1995, 72(5), 1173-1179), cardiac shock (Singh et al., Postgrad Med. 1996, 72, 45-50; Witt et al., J Am Coll Cardiol. 2001, 37(7), 1765-1774; and Gaparetto et al., Int J Clin Pharmacol Res 1991, 11(2), 83-92), traumatic shock (Gasparetto et al., Int J Clin Pharmacol Res 1991, 11(2), 83-92), cardiovascular disease (Fugh-Berman A, Prev Cardiology 2000, 3, 24-32; and Kendler B S, Prog Cardiovasc Nurs 1997, 12(3), 3-23), pain associated with sciatica (Memeo et al., Clin Drug Investig 2008, 28(8), 495-500), hyperthyroidism (Benvenga et al., J Clin Endocrinol Metab 2001, 86(8), 3579-3594), Alzheimer's disease (Thal et al., Neurology 1996, 47, 705-711; and Kidd P M, Ahern Med Rev 2008, 13(2), 85-115), hypoxia-induced oxidative stress (Dutta et al., Exp Physiol 2008, 93(10), 1139-46), chronic fatigue syndrome (Plioplys et al., Neuropsychobiology 1997, 35(1), 16-23), osteopenia (Hooshmand et al., 2008, 15(8), 595-601), osteoporosis (Hooshmand et al., 2008, 15(8), 595-601), disorders resulting from or associated with hemodialysis (Sakurabayashi et al., Cir J 2008, 72(6), 926-31), geriatric depression (Pettegrew et al., Mol Psychiatry 2000, 5, 616-632), oocyte cytoskeleton damage (Mansour et al., Fertil Steril epub Apr. 2, 2008), embryo apoptosis (Mansour et al., Fertil Steril epub Apr. 2, 2008), isotretinoin induced hepatoxicity (Georgala et al., J Eur Acad Dermatol Venereol 1999, 13(3), 205-209), doxorubicin induced toxicity (Neri et al., Anticancer Res 1986, 6(4), 659-62), valproate induced hepatotoxicity (Jung et al., Am J Emerg Med 2008, 26(3), 388 e3-4; and Sugimoto et al., Jpn J Psychiatry Neurol 1990, 44(2), 387-8), end stage renal disease (Tanner et al., J Pediatr 2007, 150(6), 631-4), peripheral arterial disease (Hiatt W R, Curr Drug Targets Cardiovasc Haematol Disord 2004, 4(3), 227-31; and Allegra et al., Ann Vasc Surg 2008, 22(4), 552-8), hepatic encephalopathy (Malaguarnera et al., Dig Dis Sci epub Mar. 21, 2008), physical fatigue (Davis et al., Curr Pain Headache Rep 2006, 10(4), 260-9; and Malaguarnera et al., Am J Clin Nutr 2007, 86(6), 1738-44), mental fatigue (Davis et al., Curr Pain Headache Rep 2006, 10(4), 260-9; and Malaguarnera et al., Am J Clin Nutr 2007, 86(6), 1738-44), primary carnitine deficiency syndromes (Hou J W, Chang Gung Med J 2002, 25(12), 832-7; and Pons et al., J Child Neurol 1995, 10 Suppl 2, S8-24), systematic carnitine deficiencies (De Vivo D C, Brain Dev 1993, 15(1), 1-22), secondary carnitine deficiency states (De Vivo et al., Epilepsia 1998, 39(11), 1216-25; and Pons et al., J Child Neurol 1995, 10 Suppl 2, S8-24), organic acidurias (Wajner et al., J Inherit Metab Dis 2004, 27(4), 427-48), leptin resistance (Iossa et al., J Nutr 2002, 132(4), 636-43), suboptimal energy partitioning (Iossa et al., J Nutr 2002, 132(4), 636-43), and disorders ameliorated by providing neuroprotection (Thangasamy et al., Biogerontology epub Jul. 16, 2008).

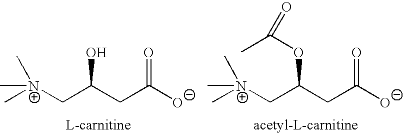

L-carnitine is metabolized by enterobacteria in the gastrointestinal track to trimethylamine and γ-butyrobetaine (Rebouche et al., Biochemistry 1984, 23, 6422-6426). Trimethylamine then undergoes extensive N-oxidation to trimethylamine-N-oxide in the liver (Lange et al., Biochem Pharmacol 1998, 56, 1005-1012). These metabolites have uncertain physiologic and toxicologic significance (Bain et al., J Clin Pharmacol 2006, 46, 1163-70). Transient nausea and vomiting have been observed with carnitine administration. Less frequent adverse reactions are body odor, nausea, and gastritis. Additionally, seizures have been reported to occur in patients, with or without pre-existing seizure activity, receiving either oral or intravenous L-carnitine.

### Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C-¹H bond. If a C-¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C-¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects.

Deuterium (2H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

L-carnitine and ALCAR are fatty acid transporters. Additionally, L-carnitine and ALCAR can remove toxic acyl-CoA compounds. The carbon-hydrogen bonds of L-carnitine and ALCAR contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could effect the pharmacokinetic, pharmacologic and/or toxicologic profiles of L-carnitine and ALCAR in comparison with L-carnitine and ALCAR having naturally occurring levels of deuterium.

Based on discoveries made in our laboratory, as well as considering the literature, L-carnitine and ALCAR, depending on the strain of bacteria, is metabolized by oxidation at various sites on the carbon backbone. The current approach has the potential to prevent metabolism at these sites. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. Since L-carnitine and ALCAR are metabolized by enterobacteria found in the gut of a subject, differences in enterobacteria strains and/or population densities can exacerbate interpatient variability. Further, some disorders, such as carnitine deficiency syndromes, are best treated when the subject is medicated around the clock or for an extended period of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of L-carnitine and ALCAR and attenuate interpatient variability.

Novel compounds and pharmaceutical compositions, certain of which have been found to modulate fatty acid transportation and/or remove toxic acyl-CoA compounds have been discovered, together with methods of synthesizing and using the compounds, including methods for the treatment of fatty acid transport-mediated disorders and/or toxic acyl-CoA compound-mediated disorders in a patient by administering the compounds as disclosed herein.

In certain embodiments of the present invention, compounds have structural Formula I:

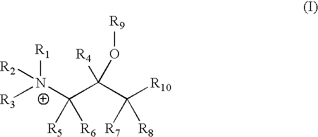

or a pharmaceutically acceptable salt, solvate, or prodrug thereof, wherein:

R₁-R₃ are independently selected from the group consisting of —CH₃, —CDH₂, —CD₂H and —CD₃;

R₄-R₈ are independently selected from the group consisting of deuterium and hydrogen;

R₉ is selected from the group consisting of deuterium, hydrogen, —COCH₃, —COCDH₂, —COCHD₂, and —COCD₃;

R₁₀ is independently selected from the group consisting of —CO₂H, —CO₂D, and —CO₂⁻¹; and

at least one of R₁-R₁₀ is deuterium or contains deuterium.

In certain embodiments the compound of Formula I is not selected from the group consisting of:

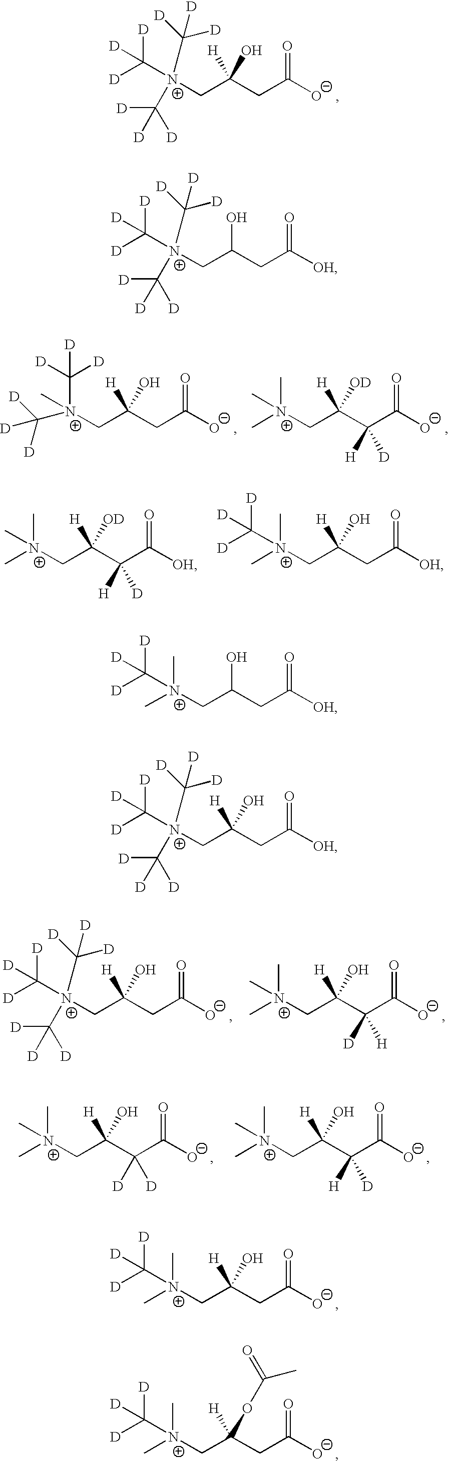

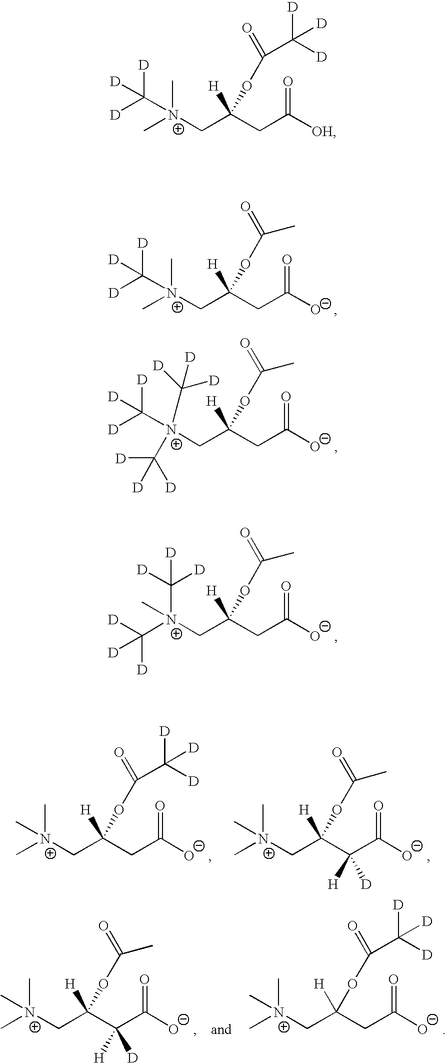

In certain embodiments the compound disclosed herein is substantially a single enantiomer, a mixture of about 90% or more by weight of the L-enantiomer and about 10% or less by weight of the D-enantiomer, a mixture of about 90% or more by weight of the L-enantiomer and about 10% or less by weight of the D-enantiomer, substantially an individual diastereomer, or a mixture of about 90% or more by weight of an individual diastereomer and about 10% or less by weight of any other diastereomer.

Certain compounds disclosed herein may possess useful fatty acid transport modulating activity and/or useful removal of toxic acyl-compound modulating activity, and may be used in the treatment or prophylaxis of a disorder in which fatty acid transport and/or toxic acyl-compounds plays an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating fatty acid transport and/or toxic acyl-CoA compound removal. Other embodiments provide methods for treating a fatty acid transport-mediated disorder, and/or a toxic acyl-CoA compound-mediated disorder, comprising administering to said patient a therapeutically effective amount of a compound or composition according to the present invention. Also provided is the use of certain compounds disclosed herein for use in the manufacture of a medicament for the prevention or treatment of a disorder ameliorated by the modulation of fatty acid transport, and/or removal of toxic acyl-CoA compounds.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The singular forms “a”, “an”, and “the” may refer to plural articles unless specifically stated otherwise.

The term “about”, as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

When ranges of values are disclosed, and the notation “from n₁ . . . to n₂” or “n₁-n₂” is used, where n₁ and n₂ are the numbers, then unless otherwise specified, this notation is intended to include the numbers themselves and the range between them. This range may be integral or continuous between and including the end values.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium”, when used to describe a given position in a molecule such as R₁-R₁₀ or the symbol “D”, when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S”, depending on the configuration of substituents around the chiral carbon atom. It should be understood that the invention encompasses all stereochemical isomeric forms, including diastereomeric, enantiomeric, and epimeric forms, as well as D-isomers and L-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease”, “syndrome”, and “condition” (as in medical condition), in that all reflect an abnormal condition of the human or animal body or of one of its parts that impairs normal functioning, is typically manifested by distinguishing signs and symptoms.

The terms “treat”, “treating”, and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent”, “preventing”, and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “fatty acid transport” refers to a compound which can bind to and form a complex with long-chain acyl groups from fatty acids. This complex can then transport the fatty acids into the mitochondrial matrix, so that they can be broken down through β-oxidation to acetate to obtain usable energy via the citric acid cycle.

The term “toxic acyl-CoA compounds” refers to compounds which normally would be converted into acetyl CoA, but instead accumulate and negatively impact the maintenance of the free coenzyme A pool. The accumulation of toxic acyl-CoA can be potentially toxic and inhibit important metabolic pathways, such as inhibiting the conversion of pyruvate to acetyl-CoA by inhibition of pyruvate dehydrogenase.

The term “fatty acid transport-mediated disorder”, refers to a disorder that is characterized by abnornal fatty transport activity. A fatty acid transport-mediated disorder may be completely or partially mediated by modulating fatty acid transport. In particular, a fatty acid transport-mediated disorder is one in which modulation of fatty acid transport results in some effect on the underlying disorder e.g., administration of a fatty acid transport modulator results in some improvement in at least some of the patients being treated.

The term “fatty acid transport modulator”, refers to the ability of a compound disclosed herein to alter fatty acid transport. A fatty acid transport modulator may increase fatty acid transport, may increase or decrease fatty acid transport depending on the concentration of the compound exposed to the fatty acid transport system, or may decrease fatty acid transport. Such activation or inhibition may be contingent on the occurrence of a specific event, such as activation of a signal transduction pathway, and/or may be manifest only in particular cell types. The term “fatty acid transport modulator” also refers to altering fatty acid transport by increasing or decreasing the probability that a complex forms between a fatty acid transporter and a natural binding partner. A fatty acid transport modulator may increase the probability that such a complex forms between the fatty acid transporter and the natural binding partner, may increase or decrease the probability that a complex forms between the fatty acid transporter and the natural binding partner depending on the concentration of the compound exposed to the fatty acid transporter, and or may decrease the probability that a complex forms between fatty acid transporter and the natural binding partner.

The term “modulation of fatty acid transport” or “modulate fatty acid transport” refers to altering fatty acid transport by administering a fatty acid transport modulator.

The term “toxic acyl-CoA compound-mediated disorder,” refers to a disorder that is characterized by toxic acyl-CoA compounds. A toxic acyl-CoA compound-mediated disorder may be completely or partially mediated by modulating the removal of toxic acyl-CoA compounds. In particular, a toxic acyl-CoA compound-mediated disorder is one in which modulating the removal of toxic acyl-CoA compound results in some effect on the underlying disorder e.g., administration of compound which removes toxic acyl-CoA results in some improvement in at least some of the patients being treated.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier”, “pharmaceutically acceptable excipient”, “physiologically acceptable carrier”, or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient”, “active compound”, and “active substance” refer to a compound, which is administered, alone or in combination with one or more pharmaceutically acceptable excipients or carriers, to a subject for treating, preventing, or ameliorating one or more symptoms of a disorder.

The terms “drug”, “therapeutic agent”, and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function is not to modify the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The compounds disclosed herein can exist as therapeutically acceptable salts. The term “pharmaceutically acceptable salt”, as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed., (Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 3 g/day. Tablets or other forms of presentation provided in discrete units may conveniently contain an amount of one or more compounds which is effective at such dosage or as a multiple of the same, for instance, units containing 5 mg to 500 mg, usually around 10 mg to 330 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating a fatty acid transport-mediated disorder, and/or toxic acyl-CoA compound-mediated disorder, comprising administering to a subject having or suspected of having such a disorder, a therapeutically effective amount of a compound as disclosed herein or a pharmaceutically acceptable salt, solvate, or prodrug thereof.

Fatty acid transport-mediated disorders, and toxic acyl-CoA compound-mediated disorders, include, but are not limited to hyperammonemic encephalopathy, PKU, diabetes, Peyronie's disease, Parkinson's disease, intermittent claudication, alcohol-related liver disease, dementia, Down's syndrome, male infertility, septic shock, cardiovascular shock, traumatic shock, pain associated with sciatica, hyperthyroidism, Alzheimer's disease, hypoxia-induced oxidative stress, muscle fatigue, geriatric depression, osteopenia, osteoporosis, disorders resulting from or associated with hemodialysis, oocyte cytoskeleton damage, cardiac disease, embryo apoptosis, isotretinoin induced hepatoxicity, doxorubicin induced toxicity, valproate induced hepatotoxicity, end stage renal disease, peripheral arterial disease, hepatic encephalopathy, physical fatigue, chronic fatigue syndrome, mental fatigue, primary carnitine deficiency syndromes, systematic carnitine deficiencies, secondary carnitine deficiency states, organic acidurias, leptin resistance, suboptimal energy partitioning, disorders ameliorated by providing neuroprotection, and/or any disorder which can lessened, alleviated, or prevented by administering a fatty acid transport modulator and/or removing toxic acyl-CoA compounds.

In certain embodiments, a method of treating a fatty acid transport-mediated disorder and/or a toxic acyl-CoA-mediated disorder comprises administering to the subject a therapeutically effective amount of a compound as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder, (7) prevention of recurrence, or delay of decline or appearance, of abnormal alimentary or hepatic parameters as the primary clinical benefit, or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described by Li et al. Rapid Communications in Mass Spectrometry 2005, 19, 1943-1950; US 2008102535; Stevens et al., Clinical Chemistry (Washington, D.C.) 2000, 46(5), 727-729; Prieto et al., Clinical Biochemistry 2006, 39(10), 1022-1027; Moeder et al., Monatshefte fuer Chemie 2005, 136(8), 1279-1291, and references cited therein and any modifications made thereof.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al., British Journal of Clinical Pharmacology, 2000, 49, 343-351. The inhibition of the MAO_{A} isoform is measured by the method of Weyler et al., J. Biol Chem. 1985, 260, 13199-13207. The inhibition of the MAO_{B} isoform is measured by the method of Uebelhack et al., Pharmacopsychiatry, 1998, 31, 187-192.

Examples of polymorphically-expressed cytochrome P₄₅₀ isoforms in a mammalian subject include, but are not limited to, CYP2C8, CYP2C9, CYP2C19, and CYP2D6.

The metabolic activities of liver microsomes, cytochrome P₄₅₀ isoforms, and monoamine oxidase isoforms are measured by the methods described herein.

Examples of improved disorder-control and/or disorder-eradication endpoints, or improved clinical effects include, but are not limited to, increased maximum exercise time, improved peak oxygen consumption, improved cardiac output after exercise, reduced arterial and pulmonary blood pressure, increased walking distance in patients with peripheral arterial disease, and increased percentage of patients showing at least one grade improvement in their peripheral neuropathy (Drug Report for Acetyl-L-Carnitine Hydrochloride, Thompson Investigational Drug Database (Sep. 15, 2008); Drug Report for Levocarnitine, Thompson Investigational Drug Database (Sep. 15, 2008); and Drug Report for Propionyl-L-Carnitine Hydrochloride, Thompson Investigational Drug Database (Sep. 15, 2008)).

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of a fatty acid transport-mediated disorder, and/or a toxic acyl-CoA-mediated disorder. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more acetylcholinesterase inhibitors known in the art, including, but not limited to metrifonate, physostigmine, neostigmine, pyridostigmine, ambenonium, demarcarium, rivastigmine, galantamine, donepezil, tacrine, and edrophonium.

In certain embodiments, the compounds disclosed herein can be combined with dietary supplements containing medium chain triglycerides.

In certain embodiments, the compounds disclosed herein can be combined with one or more angiotensin-converting enzyme inhibitors (ACE inhibitors) known in the art, including, but not limited to, captopril, enalapril, lisinopril, perindopril, ramipril, quinapril, benazepril, cilazapril, fosinopril, trandolapril, spirapril, delapril, moexipril, temocapril, zofenopril, and imidapril.

In certain embodiments, the compounds disclosed herein can be combined with one or more Angiotensin II receptor antagonists (AIIRA) known in the art, including, but not limited to, candesartan, eprosartan, irbesartan, losartan, olmesartan, tasosartan, telmisartan, valsartan, glyceryl trinitrate, isosorbide dinitrate, isosorbide mononitrate, and molsidomin pentaerythritol tetranitrate.

In certain embodiments, the compounds disclosed herein can be combined with one or more diabetes mellitus treatments known in the art, including, but not limited to, insulin (human, beef, pork, lispro, aspart, glulisine, glargine, or detemir), phenformin, metformin, buformin, glibenclamide, chlorpropamide, tolbutamide, glibornuride, tolazamide, carbutamide, glipizide, gliquidone, gliclazide, metahexamide, glisoxepide, glimepiride, acetohexamide, glymidine, acarbose, miglitol, voglibose, troglitazone, rosiglitazone, pioglitazone, sitagliptin, vildagliptin, guar gum, repaglinide, nateglinide and exenatide.

In certain embodiments, the compounds disclosed herein can be combined with one or more HMG-CoA reductase inhibitors (statins) known in the art, including, but not limited to, atorvastatin, cerivastatin, fluvastatin, lovastatin, mevastatin, pitavastatin, pravastatin, rosuvastatin, and simvastatin.

In certain embodiments, the compounds disclosed herein can be combined with one or more steroidal drugs known in the art, including, but not limited to, aldosterone, beclometasone, betamethasone, deoxycorticosterone acetate, fludrocortisone acetate, hydrocortisone (cortisol), prednisolone, prednisone, methylprenisolone, dexamethasone, and triamcinolone, flunisolide, flucticasone, mometasone furoate, tixocortol, and budesonide.

In certain embodiments, the compounds disclosed herein can be combined with one or more platelet aggregation inhibitors known in the art, including, but not limited to acetylsalicylic acid/aspirin, aloxiprin, ditazole, carbasalate calcium, cloricromen, dipyridamole, indobufen, picotamide, triflusal, clopidogrel, ticlopidine, prasugrel, beraprost, prostacyclin, iloprost, and treprostinil.

In certain embodiments, the compounds disclosed herein can be combined with one or more of the following, isotretinoin, doxorubicin, and sodium valproate.

In other embodiments, the compounds disclosed herein can be combined with one or more chemotherapeutic agents, including, but not limited to, cyclophosphamide, methotrexate, 5-fluorouracil, doxorubicin, docetaxel, epirubicin, trastuzumab, paclitaxel, capecitabine, gemcitabine hydrochloride, and abraxane.

The compounds disclosed herein can also be administered in combination with other classes of compounds, including, but not limited to, anti-retroviral agents; CYP3A inhibitors; CYP3A inducers; protease inhibitors; adrenergic agonists; anti-cholinergics; mast cell stabilizers; xanthines; leukotriene antagonists; glucocorticoids treatments; local or general anesthetics; non-steroidal anti-inflammatory agents (NSAIDs), such as naproxen; antibacterial agents, such as amoxicillin; cholesteryl ester transfer protein (CETP) inhibitors, such as anacetrapib; anti-fungal agents, such as isoconazole; sepsis treatments, such as drotrecogin-α; steroidals, such as hydrocortisone; local or general anesthetics, such as ketamine; norepinephrine reuptake inhibitors (NRTs) such as atomoxetine; dopamine reuptake inhibitors (DARTs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochlorothiazide, flumethiazide, hydroflumethiazide, bendroflumethiazide, methylchlorothiazide, trichloromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating fatty acid transport-mediated disorders, and/or toxic acyl-CoA-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of fatty acid transport-mediated disorders, and/or toxic acyl-CoA-mediated disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in Voeffray et al., Helvetica Chimica Acta 1987, 70(8), 2058-64; Gu et al., Shenyang Huagong Xueyuan Xuebao 2006, 20(2), 154-155; Overend et al., Spectrochimica Acta 1961, 17, 1205-18; Durig, et al., Journal of Raman Spectroscopy 1994, 25(2), 189-98, which are hereby incorporated in their entirety, and references cited therein and routine modifications thereof. Compounds as disclosed herein can also be prepared as shown in any of the following schemes and routine modifications thereof.

The following schemes can be used to practice the present invention. Any position shown as hydrogen may optionally be replaced with deuterium.

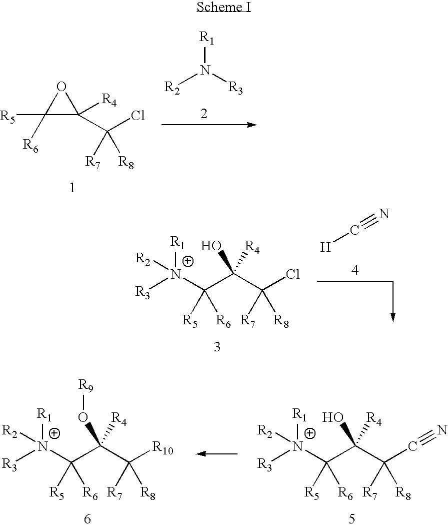

Compound 1 is reacted with compound 2 in the presence of an appropriate resolution agent, such as L-(+)-tartaric acid, in an appropriate solvent, such as methanol, to give compound 3. Compound 3 is reacted with compound 4 in the presence of an appropriate base, such as calcium hydroxide, in an appropriate solvent, such as water, to afford Compound 5. Compound 5 is treated with an appropriate acid, such as hydrochloric acid, in an appropriate solvent, such as water, to give compound 6 of Formula I (wherein R₁₀ is a carboxylic acid; and R₉ is a hydroxyl group).

Deuterium can be incorporated to different positions synthetically, according to the synthetic procedures as shown in Scheme I, by using appropriate deuterated intermediates. For example, to introduce deuterium at one or more positions of R₁-R₃, compound 2 with the corresponding deuterium substitutions can be used. To introduce deuterium at R₄-R₈, compound 1 with the corresponding deuterium substitutions can be used.

Deuterium can be incorporated to various positions having an exchangeable proton, such as the hydroxyl or carboxyl O—H groups, via proton-deuterium equilibrium exchange. For example, to introduce deuterium at R₉ and R₁₀, these protons may be replaced with deuterium selectively or non-selectively through a proton-deuterium exchange method known in the art.

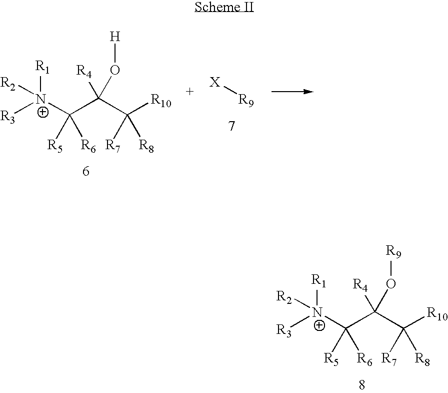

Compound 6 is reacted with compound 7 (wherein X is an appropriate acyl activating group, such as chlorine or an anhydride; and R₉ is an acetyl group), in an appropriate solvent, such as acetic acid, at an elevated temperature to afford compound 8 of Formula I (wherein R₉ is an acetyl group).

Deuterium can be incorporated to different positions synthetically, according to the synthetic procedures as shown in Scheme II, by using appropriate deuterated intermediates. For example, to introduce deuterium at one or more positions of R₁-R₈, compound 6 with the corresponding deuterium substitutions can be used. To introduce deuterium at R₉, compound 7 with the corresponding deuterium substitutions can be used.

Deuterium can be incorporated to various positions having an exchangeable proton, such as carboxyl O—H group, via proton-deuterium equilibrium exchange. For example, to introduce deuterium at R₁₀, these protons may be replaced with deuterium selectively or non-selectively through a proton-deuterium exchange method known in the art.

The following compounds can generally be made using the methods described above. It is expected that these compounds when made will have activity similar to those described in the examples above.

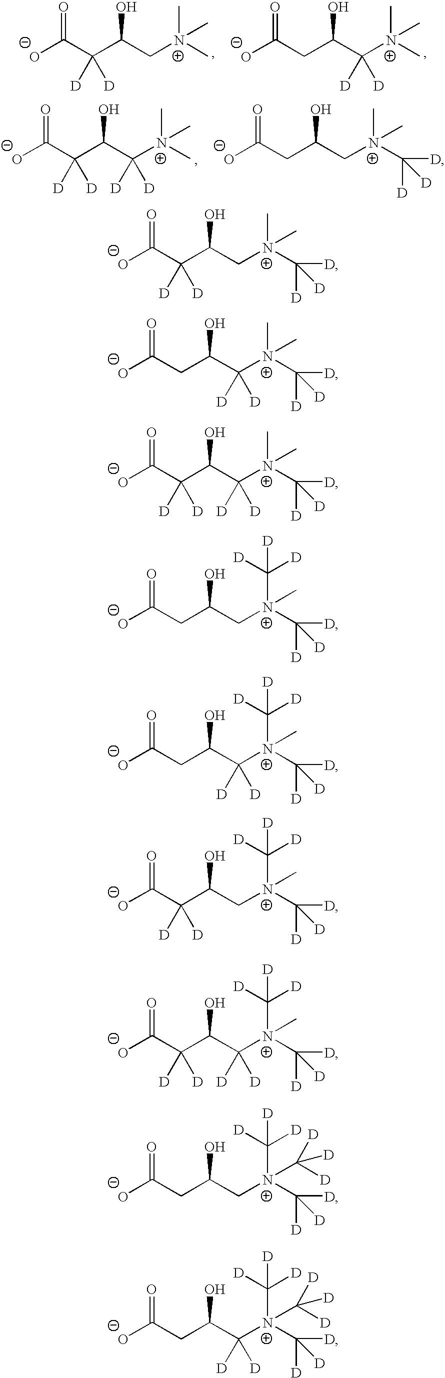

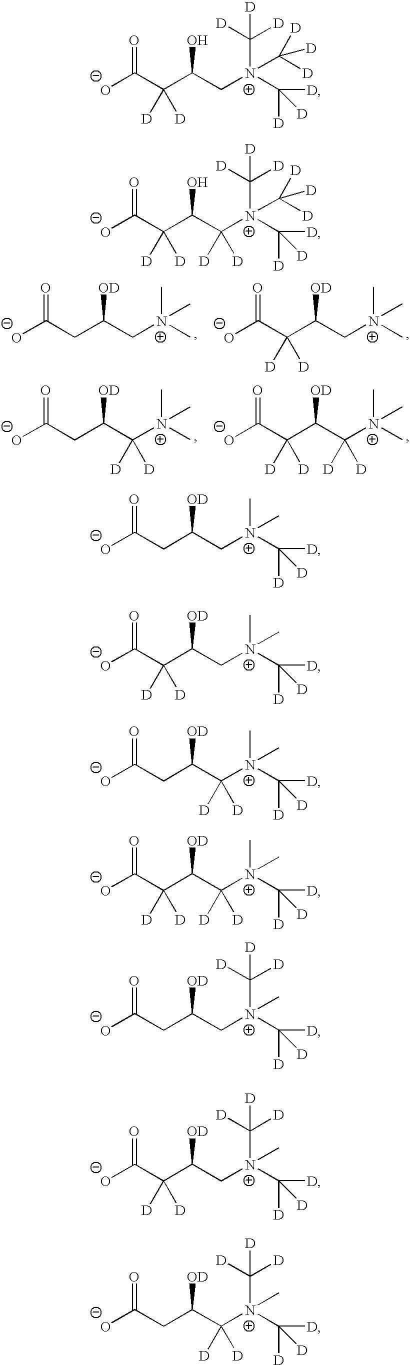

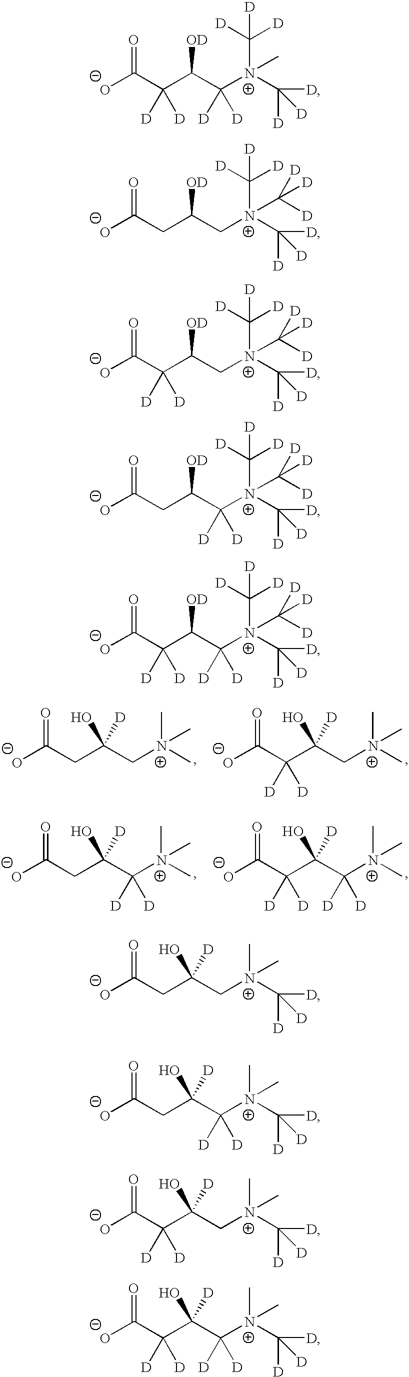

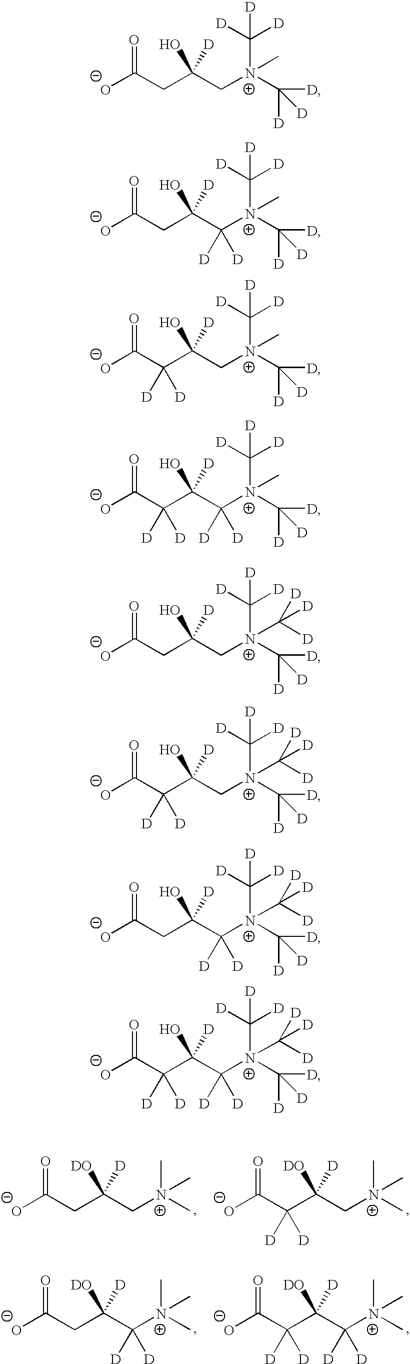

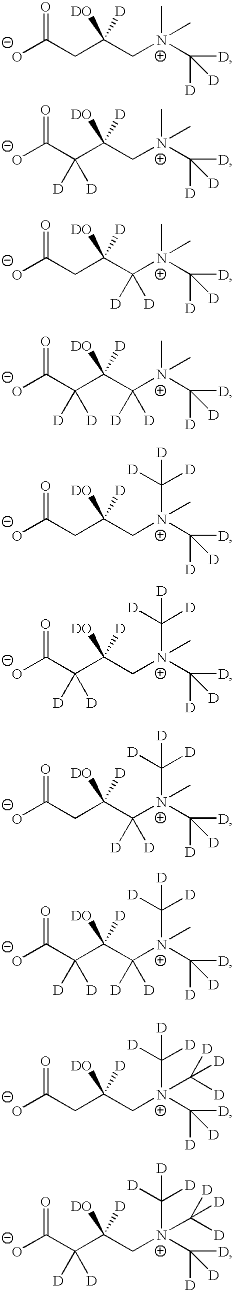

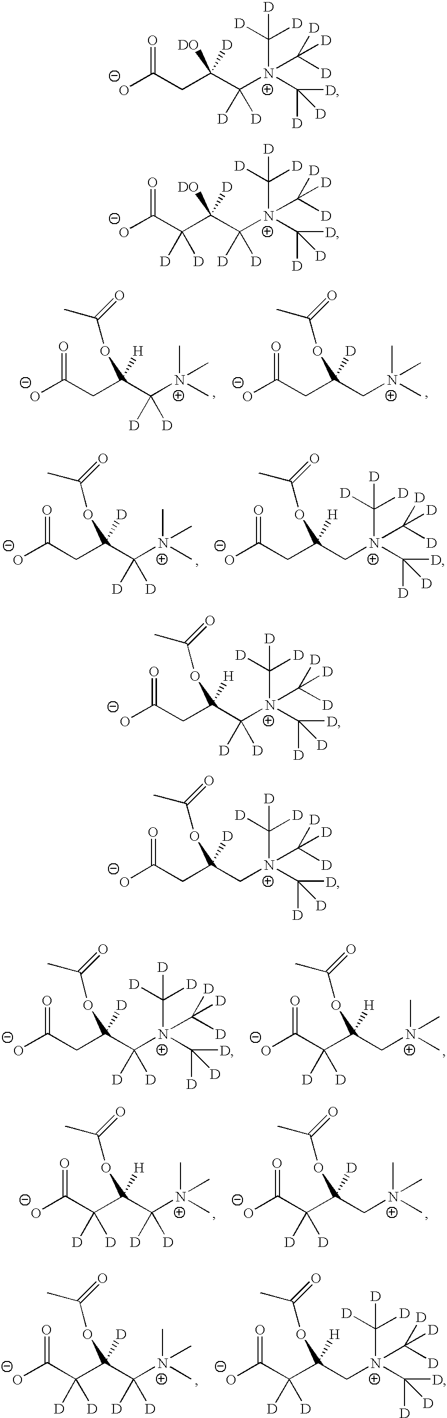

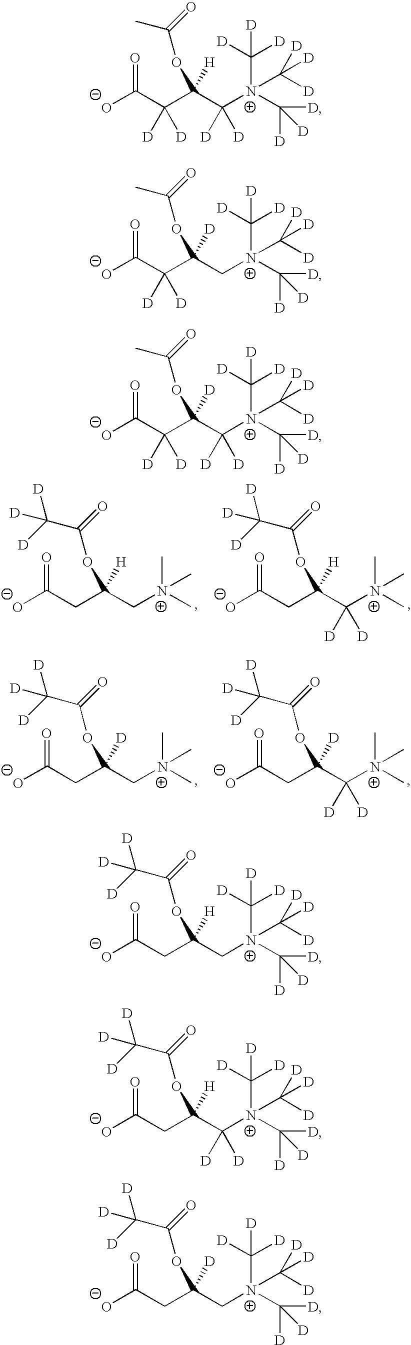

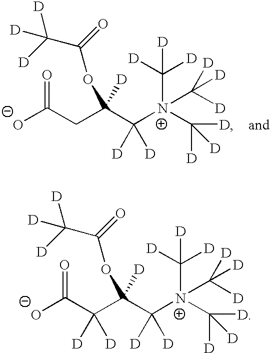

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### BIOLOGICAL ACTIVITY ASSAYS

### In Vitro Liver Microsomal Stability Assay

Liver microsomal stability assays are conducted at 1 mg per mL liver microsome protein with an NADPH-generating system in 2% sodium biphosphate (2.2 mM NADPH, 25.6 mM glucose 6-phosphate, 6 units per mL glucose 6-phosphate dehydrogenase and 3.3 mM magnesium chloride). Test compounds are prepared as solutions in 20% acetonitrile-water and added to the assay mixture (final assay concentration 5 microgram per mL) and incubated at 37° C. Final concentration of acetonitrile in the assay should be <1%. Aliquots (50 μL) are taken out at times 0, 15, 30, 45, and 60 minutes, and diluted with ice cold acetonitrile (200 μL) to stop the reactions. Samples are centrifuged at 12,000 RPM for 10 minutes to precipitate proteins. Supernatants are transferred to microcentrifuge tubes and stored for LC/MS/MS analysis of the degradation half-life of the test compounds.

### In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 minutes. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 minutes. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Monoamine Oxidase A Inhibition and Oxidative Turnover

The procedure is carried out using the methods described by Weyler et al., Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM sodium phosphate buffer, pH 7.2, containing 0.2% Triton X-100 (monoamine oxidase assay buffer), plus 1 mM kynuramine, and the desired amount of enzyme in 1 mL total volume.

### Monooamine Oxidase B Inhibition and Oxidative Turnover

The procedure is carried out as described in Uebelhack et al., Pharmacopsychiatry 1998, 31(5), 187-192, which is hereby incorporated by reference in its entirety.

### Measuring Modulatory Effects of L-Carnitine on Glucocorticoid Receptor Activity.

The procedure is carried out as described in Manoli et al., Annals of the New York Academy of Sciences 2004, 1033(Carnitine), 147-157, which is hereby incorporated by reference in its entirety.

### Assays to Measure Delays in Mitochondrial Decay

The procedure is carried out as described in Ames et al., Annals of the New York Academy of Sciences 2004, 1033(Carnitine), 108-116, which is hereby incorporated by reference in its entirety.

### Using Carnitine-Deficient Knockout Mice to Stud Effects of Carnitine Administration

The procedure is carried out as described in Takeyori et al., Annals of the Bitamin 2004, 78(11), 545-554, which is hereby incorporated by reference in its entirety.

### HPLC/MS/MS Method for Measuring and Identify in Short-Chain Acylcarnitine Isomers Due to Fatty Acid Oxidation Defects and Organic Acidemias

The procedure is carried out as described in Ferrer et al., Journal of Chromatography, B: Analytical Technologies in the Biomedical and Life Sciences 2007, 860(1), 121-126, which is hereby incorporated by reference in its entirety.

### Using an Enzymatic Reaction to Determine Free and Total Carnitine in Plasma by Spectroscopy

The procedure is carried out as described in Prieto et al., Clinical Biochemistry 2006, 39(10), 1022-1027, which is hereby incorporated by reference in its entirety.

### Various Methods for Determination of L-Carnitine and Acylcarnitines

The procedure is carried out as described in Moeder et al., Monatshefte fuer Chemie 2005, 136(8), 1279-1291, which is hereby incorporated by reference in its entirety.

### Assay for Free and Total Carnitine in Human Plasma Using Tandem Mass Spectrometry

The procedure is carried out as described in Stevens et al., Clinical Chemistry (Washington, D.C.) 2000, 46(5), 727-729, which is hereby incorporated by reference in its entirety.

From the foregoing description, one skilled in the art can ascertain the essential characteristics of this invention, and without departing from the spirit and scope thereof, can make various changes and modifications of the invention to adapt it to various usages and conditions.

What is claimed is:

1. A compound of structural Formula I 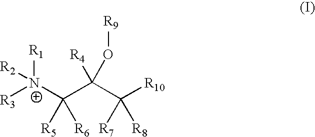
or a pharmaceutically acceptable salt thereof, wherein:
R₁-R₃ are independently selected from the group consisting of methyl, —CDH₂, —CD₂H and —CD₃;
R₄-R₈ are independently selected from the group consisting of deuterium and hydrogen;
R₉ is selected from the group consisting of deuterium, hydrogen, —COCH₃, —COCDH₂, —COCHD₂, and —COCD₃;
R₁₀ is independently selected from the group consisting of —CO₂H, —CO₂D, and —CO₂⁻;
at least one of R₁-R₁₀ is deuterium or contains deuterium; and
with the proviso that the compound cannot be selected from the group consisting of:

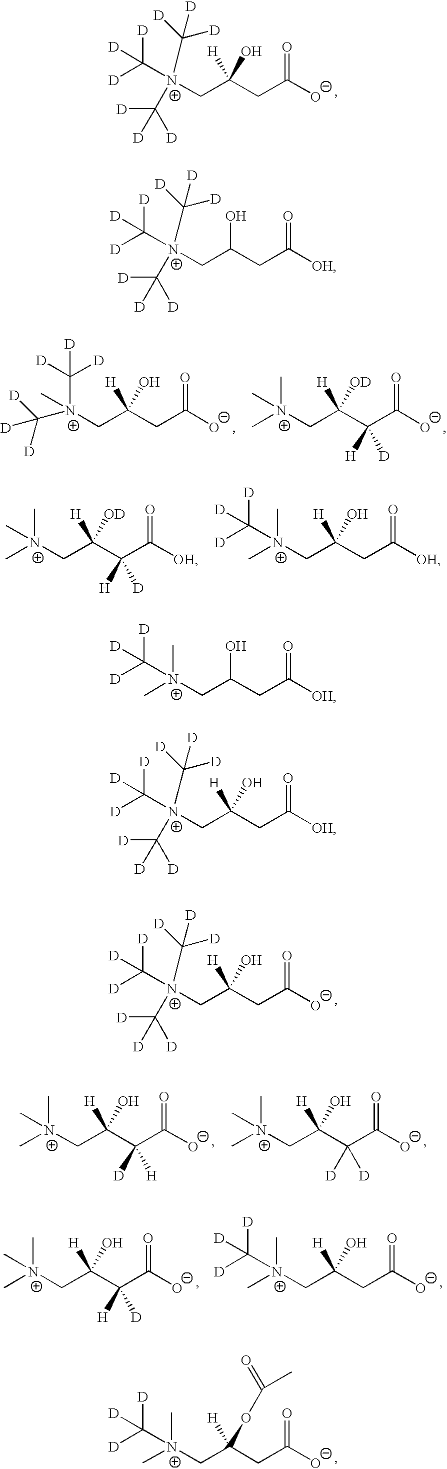

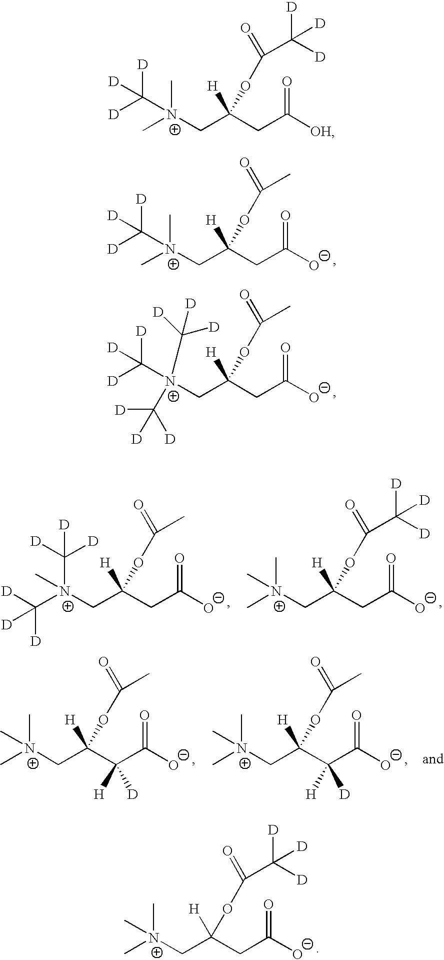

2. The compound as recited in claim 1 wherein said compound is substantially a single enantiomer, a mixture of about 90% or more by weight of the L-enantiomer and about 10% or less by weight of the D-enantiomer, a mixture of about 90% or more by weight of the L-enantiomer and about 10% or less by weight of the D-enantiomer, substantially an individual diastereomer, or a mixture of about 90% or more by weight of an individual diastereomer and about 10% or less by weight of any other diastereomer.

3. The compound as recited in claim 1 wherein at least one of R₁-R₁₀ independently has or contains deuterium enrichment of no less than about 10%.

4. The compound as recited in claim 1 wherein at least one of R₁-R₁₀ independently has or contains deuterium enrichment of no less than about 50%.

5. The compound as recited in claim 1 wherein at least one of R₁-R₁₀ independently has or contains deuterium enrichment of no less than about 90%.

6. The compound as recited in claim 1 wherein at least one of R₁-R₁₀ independently has or contains deuterium enrichment of no less than about 98%.

7. The compound as recited in claim 1 wherein said compound has a structural formula selected from the group consisting of:

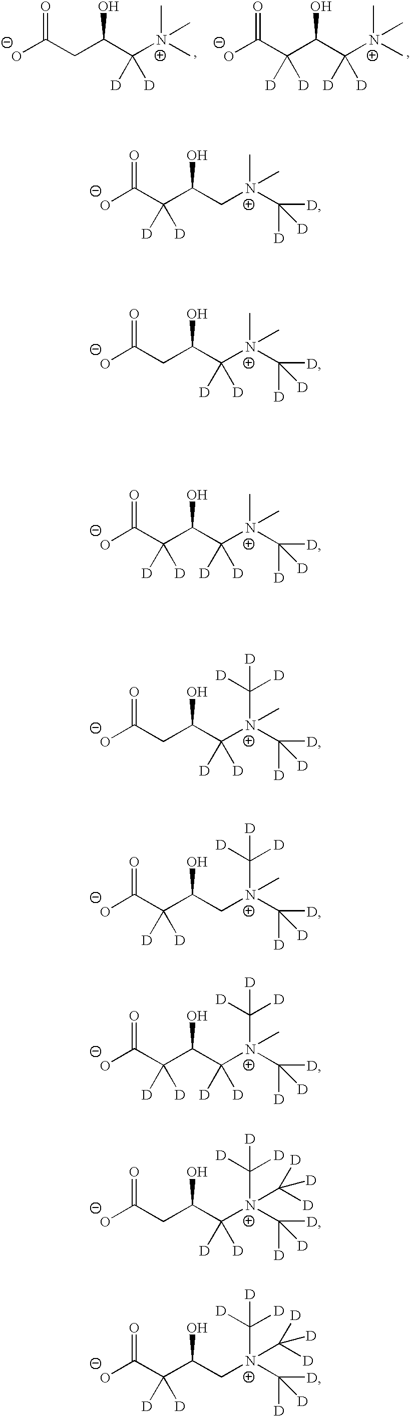

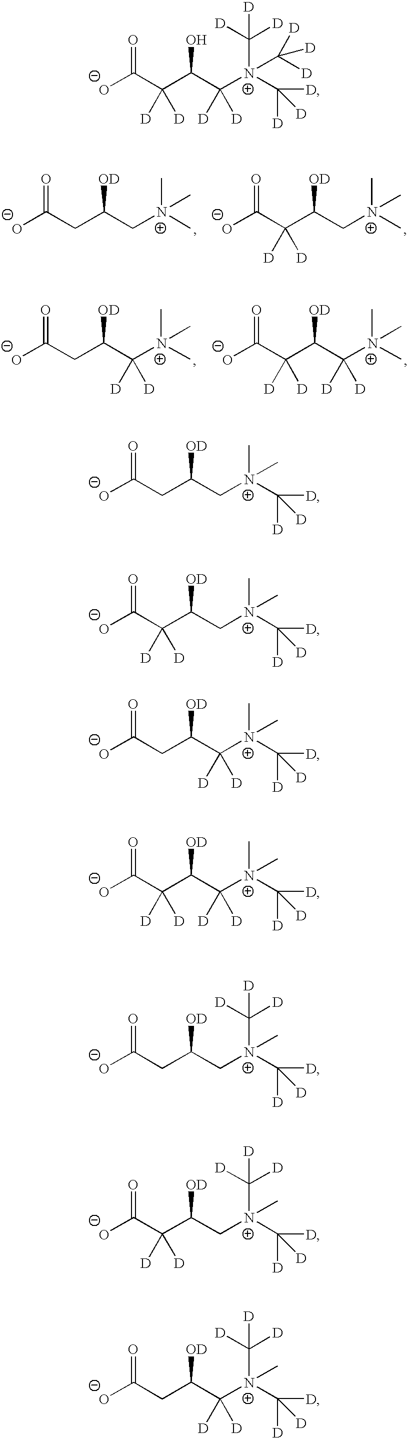

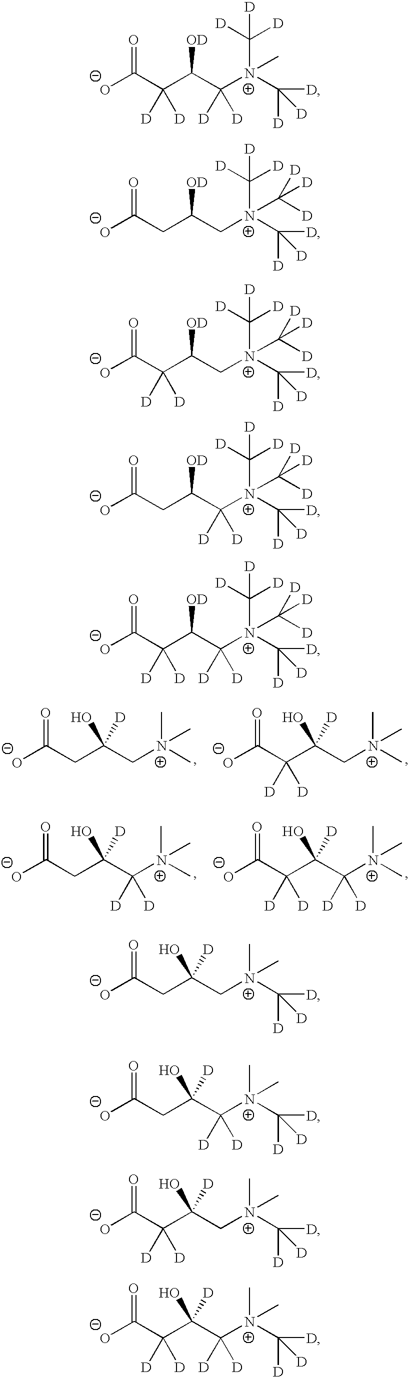

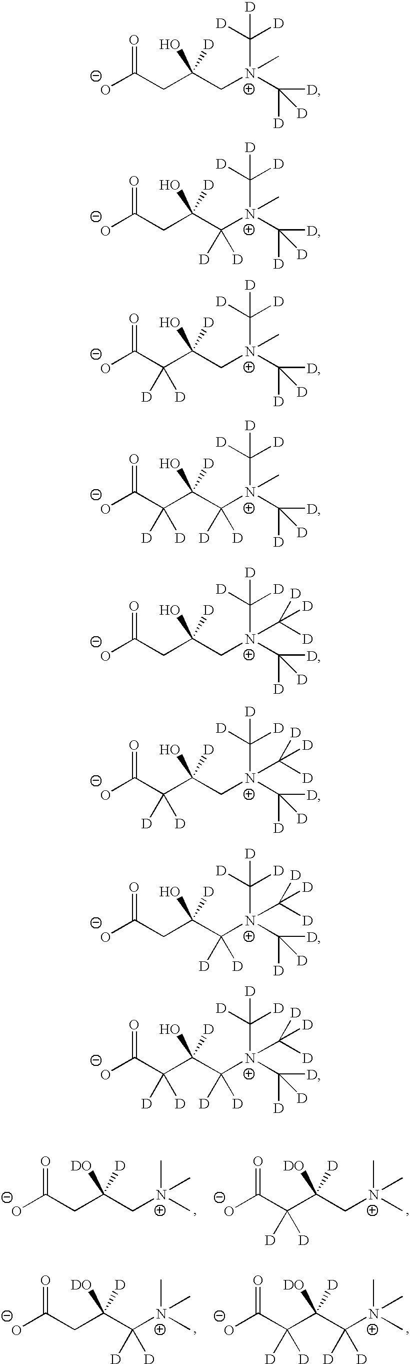

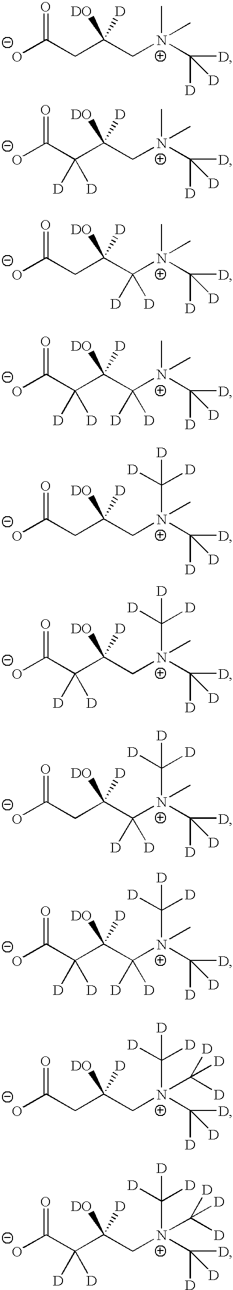

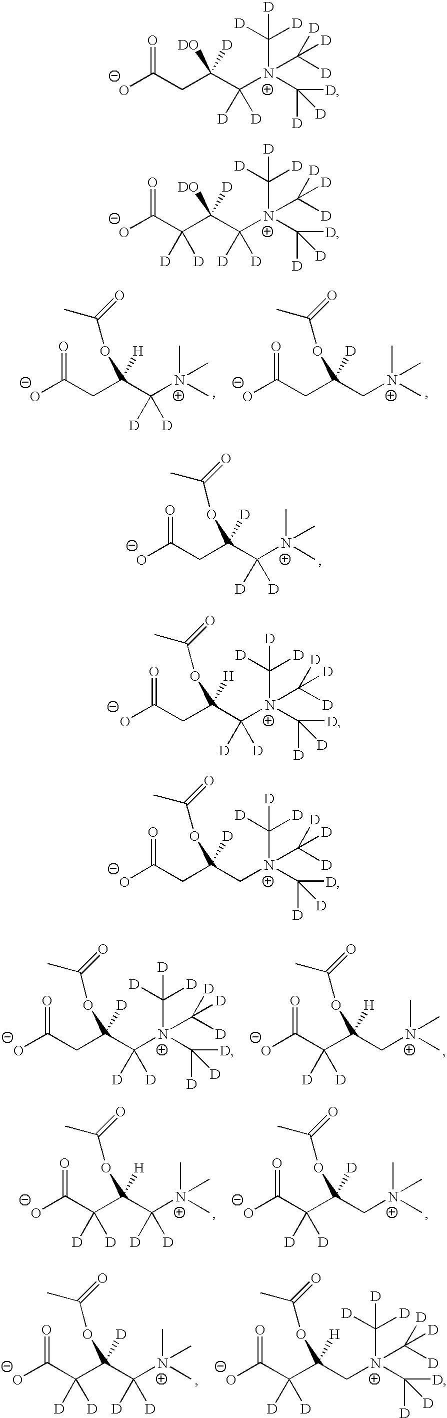

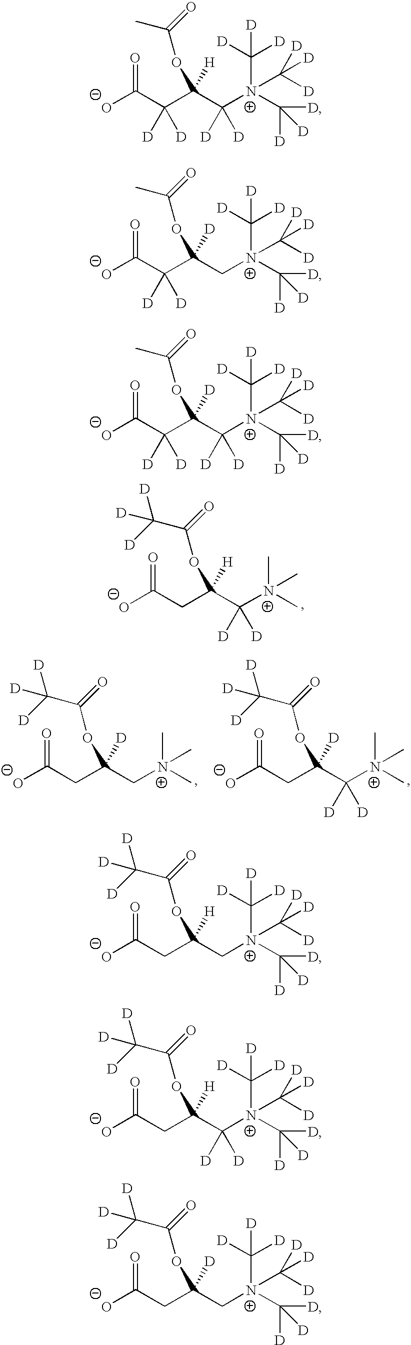

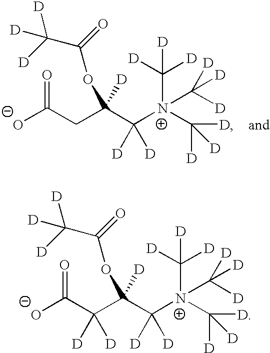

8. The compound as recited in claim 7 wherein each position represented as D has deuterium enrichment of no less than about 10%.

9. The compound as recited in claim 7 wherein each position represented as D has deuterium enrichment of no less than about 50%.

10. The compound as recited in claim 7 wherein each position represented as D has deuterium enrichment of no less than about 90%.

11. The compound as recited in claim 7 wherein each position represented as D has deuterium enrichment of no less than about 98%.

12. The compound as recited in claim 7 wherein said compound has a structural formula of:

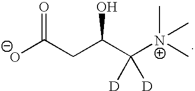

13. The compound as recited in claim 7 wherein said compound has a structural formula of:

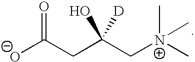

14. The compound as recited in claim 7 wherein said compound has a structural formula of:

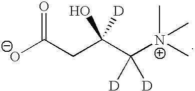

15. The compound as recited in claim 7 wherein said compound has a structural formula of:

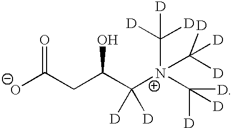

16. The compound as recited in claim 7 wherein said compound has a structural formula of:

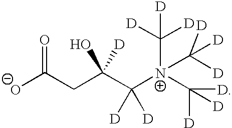

17. The compound as recited in claim 7 wherein said compound has a structural formula of:

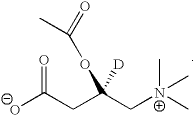

18. The compound as recited in claim 7 wherein said compound has a structural formula of:

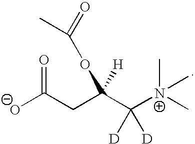

19. The compound as recited in claim 7 wherein said compound has a structural formula of:

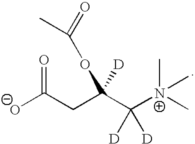

20. The compound as recited in claim 7 wherein said compound has a structural formula of:

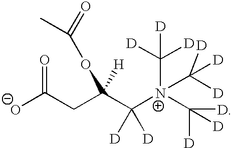

21. The compound as recited in claim 7 wherein said compound has a structural formula of:

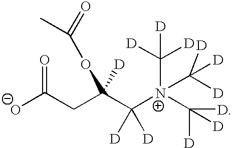

22. A pharmaceutical composition comprising a pharmaceutically acceptable carrier and a compound having structural formula I: 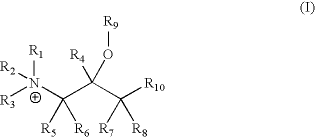
or a pharmaceutically acceptable salt thereof, wherein:
R₁-R₃ are independently selected from the group consisting of methyl, —CDH₂, —CD₂H and —CD₃;
R₄-R₈ are independently selected from the group consisting of deuterium and hydrogen;
R₉ is selected from the group consisting of deuterium, hydrogen, CH₃CO—, CDH₂CO—, CHD₂CO—, and CD₃CO—;
R₁₀ is selected from the group consisting of —CO₂H, —CO₂D, and —CO₂⁻; and
at least one of R₁-R₁₀ is deuterium or contains deuterium.

23. A method of treatment of a fatty acid transport-mediated disorder, and/or a toxic acyl-CoA compound-mediated disorder comprising the administration of a therapeutically effective amount of a compound having structural formula I: 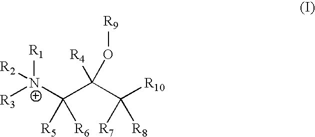
or a pharmaceutically acceptable salt thereof, wherein:
R₁-R₃ are independently selected from the group consisting of methyl, —CDH₂, —CD₂H and —CD₃;
R₄-R₈ are independently selected from the group consisting of deuterium and hydrogen;
R₉ is selected from the group consisting of deuterium, hydrogen, —COCH₃, —COCDH₂, —COCHD₂, and —COCD₃;
R₁₀ is selected from the group consisting of —CO₂H, —CO₂D, and —CO₂⁻; and
at least one of R₁-R₁₀ is deuterium or contains deuterium.

24. The method as recited in claim 23 wherein said disorder is selected from the group consisting of hyperammonemic encephalopathy, PKU, diabetes, Peyronie's disease, Parkinson's disease, intermittent claudication, alcohol-related liver disease, dementia, Down's syndrome, male infertility, septic shock, cardiovascular shock, traumatic shock, pain associated with sciatica, hyperthyroidism, Alzheimer's disease, hypoxia-induced oxidative stress, muscle fatigue, geriatric depression, osteopenia, osteoporosis, disorders resulting from or associated with hemodialysis, oocyte cytoskeleton damage, cardiac disease, embryo apoptosis, isotretinoin induced hepatoxicity, doxorubicin induced toxicity, cancer chemotherapy induced heart damage, valproate induced hepatotoxicity, end stage renal disease, peripheral arterial disease, hepatic encephalopathy, physical fatigue, chronic fatigue syndrome, mental fatigue, primary carnitine deficiency syndromes, systematic carnitine deficiencies, secondary carnitine deficiency states, organic acidurias, leptin resistance, suboptimal energy partitioning, and disorders ameliorated by providing neuroprotection.

25. The method as recited in claim 23 further comprising the administration of an additional therapeutic agent.

26. The method as recited in claim 25 wherein said additional therapeutic agent is selected from the group consisting of isotretinoin, doxorubicin, and valproate

27. The method as recited in claim 25 wherein said additional therapeutic agent is selected from the group consisting of steroidal drugs, platelet aggregation inhibitors, statins, diabetes mellitus treatments, AIIRAs, ACE inhibitors, acetylcholinesterase inhibitors, dietary supplements containing medium chain triglycerides, chemotherapeutic agents, dopamine agonists, monoamine oxidase inhibitors, dopamine prodrugs, L-dopa metabolism suppressors, adamantine-based agents, SSRIs, TCAs, barbituates, benzodiazepines, amphetamine-like stimulants, anticoagulants, thrombolytics, fibrates, bile acid sequestrants, CETP inhibitors, lipid modifying agents, NSAIDs, anti-bacterial agents, anti-fungal agents, sepsis treatments, local or general anesthetics, NRIs, DARIs, SNRIs, sedatives, NDRIs, SNDRIs, monoamine oxidase inhibitors, hypothalamic phospholipids, ECE inhibitors, opioids, thromboxane receptor antagonists, potassium channel openers, thrombin inhibitors, hypothalamic phospholipids, growth factor inhibitors, anti-platelet agents, P2Y(AC) antagonists, anticoagulants, low molecular weight heparins, Factor VIIa Inhibitors and Factor Xa Inhibitors, renin inhibitors, NEP inhibitors, vasopepsidase inhibitors, squalene synthetase inhibitors, anti-atherosclerotic agents, MTP Inhibitors, calcium channel blockers, potassium channel activators, alpha-muscarinic agents, beta-muscarinic agents, antiarrhythmic agents, diuretics, thrombolytic agents, anti-diabetic agents, mineralocorticoid receptor antagonists, growth hormone secretagogues, aP2 inhibitors, phosphodiesterase inhibitors, protein tyrosine kinase inhibitors, antiinflammatories, antiproliferatives, immunosuppressants, anticancer agents and cytotoxic agents, antimetabolites, antibiotics, farnesyl-protein transferase inhibitors, hormonal agents, microtubule-disruptor agents, microtubule-stablizing agents, plant-derived products, epipodophyllotoxins, taxanes, topoisomerase inhibitors, prenyl-protein transferase inhibitors, cyclosporins, cytotoxic drugs, TNF-alpha inhibitors, anti-TNF antibodies and soluble TNF receptors, cyclooxygenase-2 (COX-2) inhibitors, and miscellaneous agents.

28. The method as recited in claim 25 wherein said additional therapeutic agent is selected from the group consisting of steroidal drugs, platelet aggregation inhibitors, statins, diabetes mellitus treatments, AIIRAs, ACE inhibitors, acetylcholinesterase inhibitors, dietary supplements containing medium chain triglycerides, and chemotherapeutic agents.

29. The method as recited in claim 23, further resulting in at least one effect selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

30. The method as recited in claim 23, further resulting in at least two effects selected from the group consisting of:
a. decreased inter-individual variation in plasma levels of said compound or a metabolite thereof as compared to the non-isotopically enriched compound;
b. increased average plasma levels of said compound per dosage unit thereof as compared to the non-isotopically enriched compound;
c. decreased average plasma levels of at least one metabolite of said compound per dosage unit thereof as compared to the non-isotopically enriched compound;
d. increased average plasma levels of at least one metabolite of said compound per dosage unit thereof as compared to the non-isotopically enriched compound; and
e. an improved clinical effect during the treatment in said subject per dosage unit thereof as compared to the non-isotopically enriched compound.

31. The method as recited in claim 23, wherein the method effects a decreased metabolism of the compound per dosage unit thereof by at least one polymorphically-expressed cytochrome P₄₅₀ isoform in the subject, as compared to the corresponding non-isotopically enriched compound.

32. The method as recited in claim 31, wherein the cytochrome P₄₅₀ isoform is selected from the group consisting of CYP2C8, CYP2C9, CYP2C19, and CYP2D6.

33. The method as recited claim 23, wherein said compound is characterized by decreased inhibition of at least one cytochrome P₄₅₀ or monoamine oxidase isoform in said subject per dosage unit thereof as compared to the non-isotopically enriched compound.

34. The method as recited in claim 33, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

35. The method as recited in claim 23, wherein the method reduces a deleterious change in a diagnostic hepatobiliary function endpoint, as compared to the corresponding non-isotopically enriched compound.

36. The method as recited in claim 35, wherein the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

37. A compound for use as a medicament having structural Formula (I): 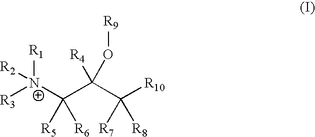
or a pharmaceutically acceptable salt thereof, wherein:
R₁-R₃ are independently selected from the group consisting of methyl, —CDH₂, —CD₂H and —CD₃;
R₄-R₈ are independently selected from the group consisting of deuterium and hydrogen;
R₉ is selected from the group consisting of deuterium, hydrogen, —COCH₃, —COCDH₂, —COCHD₂, and —COCD₃;
R₁₀ is selected from the group consisting of —CO₂H, —CO₂D, and —CO₂⁻; and
at least one of R₁-R₁₀ is deuterium or contains deuterium.

38. A compound for use in the manufacture of a medicament for the prevention or treatment of a disorder ameliorated by the modulation of fatty acid transport and/or removal of toxic acyl-CoA compounds, having structural Formula (I): 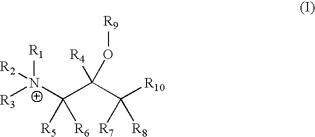
or a pharmaceutically acceptable salt thereof, wherein:
R₁-R₃ are independently selected from the group consisting of methyl, —CDH₂, —CD₂H and —CD₃;
R₄-R₈ are independently selected from the group consisting of deuterium and hydrogen;
R₉ is selected from the group consisting of deuterium, hydrogen, —COCH₃, —COCDH₂, —COCHD₂, and —COCD₃;
R₁₀ is selected from the group consisting of —CO₂H, —CO₂D, and —CO₂⁻; and
at least one of R₁-R₁₀ is deuterium or contains deuterium.
